(19) United States
(12) Patent Application Publication
(10) Pub. No.: US 2016/0235749 A1
(43) Pub. Date: Aug. 18, 2016

(54) MICRO-PARTICULATED NANOCAPSULES CONTAINING LOPINAVIR WITH ENHANCED ORAL BIOAVAILABILITY AND EFFICACY

(71) Applicant: Yissum Research Development Company of the Hebrew University of Jerusalem Ltd., Jerusalem (IL)

(72) Inventors: Simon BENITA, Tel Aviv (IL); Taher NASSAR, Kfar Tur'an (IL)

(21) Appl. No.: 15/041,610

(22) Filed: Feb. 11, 2016

Related U.S. Application Data
(60) Provisional application No. 62/115,214, filed on Feb. 12, 2015.

(51) Int. Cl. A61K 31/513; A61K 9/16; A61K 9/51

(52) U.S. Cl. CPC A61K 31/513; A61K 9/5192; A61K 9/5138; A61K 9/1682; A61K 9/1652

(57) ABSTRACT

The present disclosure provides controlled-release delivery systems for oral delivery of active agents, e.g. lopinavir, comprising micro-particulated with enhanced oral bioavailability and efficacy, which may be used for treating HIV.

### TECHNOLOGICAL FIELD

The present disclosure concerns micro-particulated nanocapsules containing lopinavir with enhanced oral bioavailability and efficacy which may be used for treating HIV.

### BACKGROUND ART

References considered to be relevant as background to the presently disclosed subject matter are listed below:
[1] Basic Information about HIV and AIDS. [11 Apr. 2012]; Available from: (http://www.cdc.gov/hiv/topics/basic/index.htm)
[2] Katzung B G, Basic & Clinical Pharmacology. 10 ed. 2007, New York: McGraw-Hill, 1088
[3] Zhang L et al., Mol Pharm. 2009, 6(6): 1766-74
[4] Sham H L et al., Antimicrob Agents Chemother. 1998, 42(12):3218-24
[5] Kumar G N et al., Pharm Res. 2004, 21(9): 1622-30
[6] Agarwal S et al., Int J Pharm. 2007, 339(1-2):139-47
[7] Cooper C L et al., Clin Infect Dis. 2003, 36(12):1585-92
[8] Youle M et al., Lipid profiles in patients on ritonavir/indinavir containing salvage regimens. 1st International Workshop on Adverse Drug Reactions and Lipodystrophy in HIV, 1999, San Diego, USA
[9] Anson B D et al., Lancet 2005, 365(9460):682-6
[10] Agarwal S et al., Int J Pharm. 2008, 359(1-2):7-14
[11] Aji Alex M R et al., Eur J Pharm Sci. 2010, 42(1-2):11-8
[12] Nassar T et al., Pharm Res. 2008, 25(9):2019-29
[13] Nassar T et al., J Control Release 2009, 133(1):77-84
[14] Saitoh H et al., Eur J Pharm Sci. 2006, 28(1-2):34-42
[15] Yokogawa K et al., Pharm Res. 1999, 16(8):1213-8
[16] Nassar T et al., Cancer Res. 2011, 71(8):3018-28
[17] Donato E M et al., J Pharm Biomed Anal. 2008, 47(3):547-52
[18] du Plooy M et al., Biol Pharm Bull. 2011, 34(1):66-70
[19] Lal R et al., Multiple dose safety, tolerability and pharmacokinetics of ABT-378 in combination with ritonavir. The 5th Conference on Retroviruses and Opportunistic Infections 1998, Chicago, USA
[20] Piscitelli S C et al., N Engl J Med. 2001, 344(13):984-96
[21] Magalhaes N S et al., J Microencapsul. 1995, 12(2): 195-205
[22] WO 207/083316

Acknowledgement of the above references herein is not to be inferred as meaning that these are in any way relevant to the patentability of the presently disclosed subject matter.

### BACKGROUND

The Acquired Immune Deficiency Syndrome (AIDS) continues to exert a devastating effect on millions of people around the world, mostly in countries with limited resources [1]. Substantial advances have been made in antiretroviral therapy, and at least four classes of antiretroviral agents are in use: nucleotide reverse transcriptase inhibitors (NRTIs); non-NRTIs (NNRTIs); protease inhibitors (PIs); and integrase inhibitors. Patient-tailored therapy, comprising 3-4 potent and effective agents derived from different drug classes are effective in reducing the viral load to an undetectable level. These protocols may require fine-tuning if resistance of the virus evolves [2]. The necessary poly-pharmacy in most HIV-infected patients requires awareness because of potential drug-drug interactions. NNRTIs and PIs are metabolized by the CYP450 enzyme system, primarily the 3A4 isoform, resulting in several pharmacokinetic (PK) complications. In addition, many of these drugs are substrates as well as inducers or inhibitors of CYP3A4 and therefore the drug-drug interactions are unpredictable. The PI, Ritonavir (RTV), with a high oral bioavailability and long plasma half-life, is a moderate P-gp and strong CYP3A inhibitor [3]. Lopinavir (LPV), an analogue of RTV, was designed to enhance the interaction with the mutated area of the HIV protease. Indeed, LPV potently inhibits wild-type and mutated HIV proteases, prevents the replication of laboratory and clinical strains of HIV-1 and maintains high potency against selected HIV-1 mutants following RTV treatment [4]. Unfortunately, this potent and efficient PI exhibits low oral and variable bioavailability in rats and humans when given alone owing to P-gp and MRP2 efflux and extensive pre-systemic metabolism by CYP3A4 [5,6]. This process occurs mainly on the villus tip of the enterocytes in the intestine, reducing LPV plasma levels, thereby decreasing its anti-HIV efficacy. However, co-administration of LPV with a low-dose of RTV markedly improves its oral absorption and prolongs its half-life resulting in increased plasma levels of LPV. High LPV plasma concentrations, relatively to the IC₅₀, are the key for successful treatment [7]. The combination of LPV and RTV has been proven to be effective and durable. The LPV/RTV combination is marketed under the tradenames of Kaletra® or Aluvia®. However, a few drawbacks are associated with RTV treatment: P-gp efflux modulation may cause increased toxic effects by inhibiting the efflux of unsafe molecules that are normally extruded; side effects such as taste and gastrointestinal disturbances [8] may decrease patient compliance; and severe triglyceride and cholesterol abnormalities have been attributed to the presence of RTV. Finally, it has been reported that PIs can prolong the QT interval; moreover, CYP3A inhibition raises the LPV and RTV concentrations, enhancing the likelihood of QT prolongation [9]. Therefore, the use of LPV at an effective controlled therapeutic dose, without the CYP3A inhibition will relatively decrease the risk of QT lengthening.

Various strategies have been employed to create efficient therapeutic delivery systems of LPV without concomitant administration of RTV. Agarwal et al. [10] synthesized peptide prodrugs of LPV to evade the first-pass metabolism and efflux of LPV (in-vivo studies were not reported). Aji Alex et al. [11] encapsulated LPV in solid lipid nanoparticles (SLN) targeted for intestinal lymphatic vessels and increased the AUC in the lymphatic system 2-folds compared to the LPV solution. Comparative encapsulation of Kaletra® into SLN was not reported. Indeed, there is an interest and a need to develop safer formulations of antiretroviral agents, especially for children.

### GENERAL DESCRIPTION

The present invention concerns incorporation of lopinavir into biodegradable nanocapsules (NCs) embedded in gastro-resistant bio-adhesive microparticles (MCPs).

Although several MCPs containing therapeutics-carrying nanocapsules are known [12-16], the presently disclosed invention provides MCPs which embed a plurality of nanocapsules loaded with lopinavir that significantly improve absorption and controlled-release of lopinavir when administered orally, without co-administration of other anti-viral agents, such as ritonavir.

Thus, in one of its aspect, the present disclosure provides a controlled-release delivery system for oral delivery of lopinavir, the delivery system comprising at least microparticle formed of a hydrophilic polymeric matrix, and embedding at least one nanocapsule, the at least one nanocapsule comprises a core comprising lopinavir solubilized in at least one oil, and a shell comprising a hydrophobic polymer, the weight ratio of the lopinavir to the hydrophobic polymer being at least 1:1.5.

In other words, the delivery system of the present disclosure comprises at least one nanocapsule (in some embodiments a plurality of nanocapsules), carrying the active agent (lopinavir) within their oil core. The nanocapsule(s) is embedded within a microparticle, such that a delivery system suitable for oral delivery of the active agent is formed, providing protection and stabilization of the active agent from the conditions in the gastrointestinal (GI) tract and controlled release of the active agent upon exposure to suitable conditions (e.g. upon increase in pH).

Lopinavir is a protease inhibitor, having the chemical name (2S)—N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide. In the context of the present disclosure, any referral to lopinavir is meant to also encompass any suitable pharmaceutical salt of lopinavir.

In some embodiments, the core consists of lopinavir and the at least one oil, optionally further consisting at least one surfactant.

In some embodiments, the delivery system may or may not contain other protease inhibitors, such as ritonavir.

In other embodiments, the delivery system does not contain other protease inhibitors. According to such embodiments, the delivery system does not contain ritonavir.

In the delivery system of the present disclosure, lopinavir is in an encapsulated form; i.e. in the form of a microparticle into which a plurality of nanocapsules (i.e. one or more nanocapsules), is embedded, at least one of said plurality of nanocapsules containing lopinavir. The term “encapsulation” (or any lingual variation thereof) refers to, e.g., the containment lopinavir in a nanocapsule, or the containment of at least one nanocapsule within a microparticle as will be further explained.

The nanocapsule (NC) is a particulate carrier having distinct core and shell components, which is biocompatible and sufficiently resistant to chemical and/or physical destruction, such that a sufficient amount of the nanocapsules remains substantially intact after being released from the microparticle following oral administration into the human or animal body and for a sufficient period of time to reach the desired target organ (tissue). Generally, the nanocapsules are spherical in shape, having an average diameter of up 500 nanometers (nm).

In some embodiments, the averaged diameter of a nanocarrier is at least about 40 nm, at times between 40 and 500 nm.

In some embodiments, the averaged diameter of a nanocarrier is between about 50 and 500 nm, 100 and 500 nm, 150 and 500 nm, between about 200 and 500 nm, between about 250 and 500 nm or even between about 300 and 500 nm. In other embodiments, the averaged diameter is between about 100 and 450 nm. In other embodiments, the averaged diameter is between about 100 and 400 nm. In some other embodiments, the averaged diameter is between about 150 and 250 nm.

It should be noted that the averaged diameter of nanocapsules may be measured by any method known to a person skilled in the art. The term “averaged diameter” refers to the arithmetic mean of measured diameters, wherein the diameters range ±25%, ±15%, ±10%, or ±5% of the mean. Where the nanocapsules are not spherical, the term refers to the effective average diameter being the largest dimension of the nanocapsule.

A nanocapsule in a delivery system of the invention is constituted by a core comprising lopinavir solubilized in at last one oil, while the shell is formed of a hydrophobic polymer, that forms a nanocapsule about the oily core.

As a person versed in the art would understand, the “hydrophilicity” of the materials is a characteristic of materials exhibiting affinity for water, while the “hydrophobic” materials possess the opposite response to water. The material hydrophobicity or hydrophilicity may be due to the material intrinsic behaviors towards water, or may be achieved (or tuned) by modifying the material by one or more of cross-linking said material, derivatization of the material, charge induction to said material (rendering it positively or negatively charged), complexing or conjugating said material to another material and by any other means known in the art.

Thus, in accordance with the present invention, the selection of a material may be based on the material intrinsic properties or based on the material's ability to undergo such aforementioned modification to render it more or less hydrophobic or hydrophilic.

As noted above, the encapsulation shell comprises, and at time constituted by, a hydrophobic material, typically at least one hydrophobic polymer. In some embodiments, the encapsulation shell comprises lactic acid, poly(D,L-lactic-co-glycolic acid) (PLGA), poly(D,L-lactic acid) (PLA), poly(ε-caprolactone), poly(2-dimethylamino-ethylmethacrylate) homopolymer, poly(2-dimethylamino-ethylmethacrylate)-b-poly(ethyleneglycol)-α-methoxy-ω-methacrylate copolymers, polycyanoacrylates and combinations thereof and their PEGylated derivatives.

According to other embodiments, the encapsulation shell comprises lactic acid, poly(D,L-lactic-co-glycolic acid) (PLGA) and combinations thereof, including mixtures with PEGylated derivatives thereof. According to some other embodiments, the encapsulation shell comprises D,L-lactic-co-glycolic acid, where the D- and L-lactic acid forms are in approximately equal ratio, including mixtures with PEGylated derivatives thereof.

In some embodiments, the hydrophobic polymer has a molecular weight of between about 2,000 and 100,000 Da.

As noted above, the weight ratio of the lopinavir to the hydrophobic polymer is at least 1:1.5. In some embodiments, the ratio (w/w) of lopinavir to the hydrophobic polymer may be between about 1:1.5 and 1:3. In such embodiments, the ratio (w/w) of lopinavir to the hydrophobic polymer may be between about 1:1.5 and 1:2.8, between about 1:1.5 and 1:2.6, between about 1:1.5 and 1:2.6, between about 1:1.5 and 1:2.4, or even between about 1:1.5 and 1:2.2. In other such embodiments, the ratio (w/w) of lopinavir to the hydrophobic polymer may be between about 1:1.6 and 1:3, between about 1:1.7 and 1:3, between about 1:1.8 and 1:3, between about 1:1.9 and 1:3, or even between about 1:2 and 1:3. In some other embodiments, the ratio (w/w) of lopinavir to the hydrophobic polymer may be between about 1:1.7 and 1:2.5, between about 1:1.8 and 1:2.4, or even between about 1:1.9 and 1:2.3. According to some embodiments, the ratio (w/w) of lopinavir to the hydrophobic polymer is about 1:2.

It is of note that in the delivery system of the present disclosure, the weight ratio between the lopinavir and the hydrophobic polymer is one of the primary parameters controlling and increasing the thickness of the nanocapsules' shell. At times, other weight ratios to be discussed herein (i.e. in addition to the weight ratio between lopinavir and the hydrophobic polymer) may also have a role in determining such thickness.

In the core, the lopinavir is present in a solubilized form in at least oil. The oil may be constituted by single oil or a mixture of two or more suitable oils. In some embodiments, the oil may be selected from long chain vegetable oils (such as corn oil, peanut oil, coconut oil, castor oil, sesame oil, soybean oil, perilla oil, sunflower oil, argan oil and walnut oil), ester oils, higher liquid alcohols, liquid fatty acids, medium chain triglycerides, natural fats and oils, and silicone oils. According to other embodiments, the oil may be selected from liquid fatty acids and esters thereof. According to such embodiments, the oil may be selected from oleic acid, octanoic acid or mixtures thereof.

In order to facilitate formation of nanocapsules, the oil core may, by some embodiments, further comprise at least one surfactant.

The term surfactant should be understood to encompass any agent that is capable of lowering the surface tension of a liquid, allowing for the formation of a homogeneous mixture of at least one type of liquid with at least one other type of liquid, or between at least one liquid and at least one solid. Thus, surfactants in the oil core may be used to control the surface tension and surface interaction between the oil and its surroundings, thereby assisting in the process of forming the nanocapsules.

Non-limiting examples of suitable surfactants are oleoyl polyoxyl-6-glycerides NF (Labrafil M1944 CS, Gatefosse), and other nonionic oil surfactants having a hydrophilic-lipophilic balance (HLB) value between 3 and 10, such as Brij® L4 (average Mₙ˜362), MERPOL® SE, poly(ethylene-glycol) sorbitol hexaoleate, poly(ethyleneglycol)-block-poly(propyleneglycol)-block-poly(ethyleneglycol) (average Mₙ˜5,800), Labrafil® M2125CS, Labrafil® M2130CS and Tefose 63.

In order to improve the oral bioavailability of lopinavir, the nanocapsules are further embedded (i.e. encapsulated) within a microparticle, the microparticle comprising hydrophilic polymers which enable controlled release of the nanocapsules in the gastrointestinal (GI) tract to permit control and sustained release of lopinavir by oral administration.

The term microparticle (at times also interchangeably referred to as microsphere) is meant to encompass micron- or submicron particles of a substantially uniform composition, constituted by a continuous matrix material, i.e. not featuring a distinct core/shell structure. The microparticles may be solid or semi-solid, e.g. in partially gelled form.

In some embodiments, the microparticles have an average diameter of between about 0.5 and 20 μm. In other embodiments, the microparticles have averaged diameter of between about 0.5 and 15 μm, between about 0.5 and 10 μm, or between about 0.5 and 5 μm. In further embodiments, the averaged diameter is between about 1 and 12 μm. In additional embodiments, the microparticles have averaged diameter of between 1 and 8 μm.

Within the microparticles described herein, at least one, at times a plurality of lopinavir-encapsulating nanocapsules are embedded within a hydrophilic polymeric matrix; meaning that the nanocapsules are distributed within the hydrophilic matrix material, and are substantially encased thereby. Once in the GI tract, the hydrophilic polymer permits (i.e. by dissolution, decomposition or swelling) release of the embedded nanocapsules for controlled and sustained delivery of lopinavir via the GI mucosal tissue.

The hydrophilic polymer is meant to encompass a polymeric substance, either a single polymer or a blend of polymers, which have the tendency to undergo a physical and/or chemical change, i.e. dissolution, decomposition, swelling, etc., due to interaction with aqueous surroundings. In some embodiments, the hydrophilic polymer undergoes the desired change once exposed to aqueous surroundings of a desired pH.

In some embodiments, the hydrophilic polymer is an enteric polymer (or polymer blend). Enteric polymers are typically insoluble at low pH environment, while dissolving or forming a hydrogel at higher pH surroundings. A typical pH threshold for enteric polymers is pH 4-5, which provides protection of the nanocapsule (and therefore also of the lopinavir) from undesired decomposition due to the extremely acidic conditions of the stomach at fast conditions, thereby assisting in controlled and improved oral delivery of the lopinavir.

In some embodiments, the hydrophilic polymer is selected from shellac, zien, poly(methacrylic acid), ethyl acrylate, polyols, polycarbohydrates, hydroxypropylmethyl cellulose (HPMC), hydroxymethyl cellulose, hydroxypropylmethylcellulose phthalate (HP55), cellulose acetate phthalate, carboxy-methylcellulose phthalate, and copolymers and mixtures thereof.

In other embodiments, comprises an HPMC:Eudragit (poly(methacrylic acid)-ethyl acrylate copolymer) blend. In such an Eudragit:HPMC blend, the Eudragit has pH-dependent solubility, while HPMC is aqueous soluble irrespective of the pH.

As used herein, either in connection with the nanocapsule or the microparticle, the term “polymer” includes homopolymers, copolymers, such as for example, block, graft, random and alternating copolymers as well as terpolymers, further including their derivatives, combinations and blends thereof. In addition to the above, the term includes all geometrical configurations of such structures including linear, block, graft, random, alternating, branched structures, and combination thereof.

The polymers utilized in the construction of the nanocapsules and the microparticles are biodegradable, namely, they degrade during in vivo use. In general, degradation attributable to biodegradability involves the degradation of a biodegradable polymer into its component subunits, or digestion, e.g., by a biochemical process carried out for example by enzymes, of the polymer into smaller, non-polymeric subunits. The degradation may proceed in one or both of the following: biodegradation involving cleavage of bonds in the polymer matrix, in which case, monomers and oligomers typically result, or the biodegradation involving cleavage of a bond internal to side chain or that connects a side chain to the polymer backbone. In some embodiments, biodegradation encompasses both general types of biodegradation. The polymers are additionally biocompatible, namely, they are substantially non-toxic or lacking injurious impact on the living tissues or living systems to which they come in contact with.

The microparticle of the invention should be large enough to be able to hold at least one nanocapsule, typically a plurality of nanocapsules (i.e. two or more), yet at the same time be of a smaller enough size to be able to undergo internalization once administered.

The number of nanocapsules which are encapsulated within a single microparticle may vary depending on, e.g., the size of the nanocapsule or the relative sizes of the nanocapsule and the microparticle. Typically, each microparticle may contain between 1 and a few hundreds or even thousands of nanocapsules (being said plurality of nanocapsules).

The microcapsules may encapsulate a plurality of various nanocapsules. For example, the microparticle may contain a plurality of nanocapsules of the same polymeric material (thus having the same hydrophilic/hydrophobic properties), some of which comprising one type of oil and the other comprise another type of suitable oil. Similarly, the microparticles of the invention may contain a plurality of nanocapsules of different suitable polymeric materials, however containing each the same oil. Some of the nanocapsules may comprise varying forms of lopinavir or salts thereof, at the same or different concentration. Some of the nanocapsules may comprise other protease inhibitors (excluding ritonavir).

The microparticle, by some embodiments, may comprise between about 1 and 10 wt % of lopinavir. According to some embodiments, the microparticle may comprise between about 1 and 9 wt % of lopinavir, between about 1 and 8 wt % of lopinavir, between about 1 and 7 wt % of lopinavir, or even between about 1 and 6 wt % of lopinavir. According to other embodiments, the microparticle may comprise between about 2 and 10 wt % of lopinavir, between about 3 and 10 wt % of lopinavir, between about 4 and 10 wt % of lopinavir, or even between about 5 and 10 wt % of lopinavir.

According to some embodiments, of the delivery system of the present disclosure, the weight ratio of the lopinavir to the oil is between 1.5:4 and 1.5:6. In such embodiments, the weight ratio of the lopinavir to the oil may be between 1.5:4.2 and 1.5:6, between 1.5:4.4 and 1.5:6, between 1.5:4.6 and 1.5:6, or even between 1.5:4.8 and 1.5:6. In other such embodiments, the weight ratio of the lopinavir to the oil may be between 1.5:4 and 1.5:5.8, between 1.5:4 and 1.5:5.6, between 1.5:4 and 1.5:5.4, or even between 1.5:4 and 1.5:5.2. According to other embodiments, the weight ratio of the lopinavir to the oil may be between 1.5:4.5 and 1.5:5.5, between 1.5:4.6 and 1.5:5.4, or even between 1.5:4.8 and 1.5:5.2.

In embodiments where the core comprises at least one emulsifier, the weight ratio of the lopinavir to the emulsifier is between 1:1 and 2:1. In such embodiments, the weight ratio of the lopinavir to the emulsifier may be between 1.1:1 and 2:1, between 1.2:1 and 2:1, between 1.3:1 and 2:1, or even between 1.4:1 and 2:1. In other such embodiments, the weight ratio of the lopinavir to the emulsifier may be 1.5:1.

In other embodiments where the core comprises at least one emulsifier, the weight ratio of the hydrophobic polymer to the emulsifier is between 2:1 and 4:1. In such embodiments, the weight ratio of the hydrophobic core to the emulsifier may be between 2.2:1 and 4:1, between 2.4:1 and 4:1, between 2.6:1 and 4:1, or even between 2.8:1 and 4:1. In other such embodiments, the weight ratio of the hydrophobic polymer to the emulsifier may be between 2:1 and 3.8:1, between 2:1 and 3.6:1, between 2:1 and 3.4:1, or even between 2:1 and 3.2:1. According to other embodiments, the weight ratio of the hydrophobic polymer and the emulsifier may be between 2.2:1 and 3.8:1, between 2.4:1 and 3.6:1, or even between 2.6:1 and 3.4:1. According to some embodiments, the weight ratio of the hydrophobic polymer to emulsifier is 3:1.

In some embodiments, the weight ratio of the hydrophobic polymer to the oil is between at least 3:5. In some other embodiments, the weight ratio of the hydrophobic polymer to the oil is 3:5.

In some embodiments, in the nanocapsule:
(i) the weight ratio of the lopinavir to the oil being between 1.5:4 and 1.5:6; and/or;
(ii) the core further comprises at least one emulsifier, the weight ratio of the lopinavir to the emulsifier being between 1:1 and 2:1; and/or
(iii) the core further comprises at least one emulsifier, the weight ratio of the hydrophobic polymer to the emulsifier being between 2:1 and 4:1; and/or
(iv) the weight ratio of the hydrophobic polymer to the oil being at least 3:5.

According to some embodiments, the weight ratio of lopinavir to hydrophobic polymer to oil in the nanocapsule is 1.5:3:5.

The delivery system of the present disclosure may be in dry form (i.e. dry powder), or provided as a dispersion/suspension in a suitable pharmaceutical liquid carrier, as will be described further herein.

In another aspect of the present disclosure, there is provided a controlled-release delivery system for oral delivery of a protease inhibitor, the delivery system comprising at least microparticle formed of a hydrophilic polymeric matrix, and embedding at least one nanocapsule, the at least one nanocapsule comprises a core comprising an a protease inhibitor solubilized in at least one oil, and a shell comprising a hydrophobic polymer, the weight ratio of the protease inhibitor to the hydrophobic polymer being at least 1:1.5.

The protease inhibitor may be an antiretroviral active agent having a lop P (i.e. a partition coefficient between water and the oil) of between about 2 and 7. In some embodiments, the protease inhibitor may be selected from the group consisting of lopinavir, tipranavir (Aptivus), indinavir (Crixivan), atazanavir (Evotaz, Reyataz), saquinavir (Invirase), fosamprenavir (Lexiva), darunavir (Prezcobix, Prezista), and nelfinavir (Viracept).

According to some embodiments, the protease inhibitor is lopinavir.

By another aspect, the present disclosure provides a method of preparing the delivery system for oral delivery of lopinavir as described herein, comprising:
mixing (i) at least one oil and lopinavir with (ii) a solution of a hydrophobic polymer in an organic solvent, optionally in the presence at least one surfactant, the weight ratio of the lopinavir to the hydrophobic polymer being at least 1:1.5, to thereby form an organic phase;
adding water to said organic phase under conditions permitting the formation of a suspension of nanocapsules;
mixing said suspension of nanocapsules with an aqueous solution of at least one hydrophilic polymer to obtained a mixed suspension; and
spray drying the mixed suspension, thereby obtaining microparticles formed of said hydrophilic polymer that embed at least one nanocapsule (the nanocapsule comprising a core comprising lopinavir solubilized in at least one oil, and a shell comprising a hydrophobic polymer, the weight ratio of the lopinavir to the hydrophobic polymer being at least 1:1.5).

In the method of the present disclosure, the active agent (i.e. lopinavir), the oil and the emulsifier (when used), are first solubilized in a solvent to form a homogenous organic phase.

Appropriate organic solvents are, for example, polar solvents which can substantially solublize the hydrophobic solvent and also exhibit good solubility in water. In some embodiments, the solvent may be selected from acetone, methanol, ethanol, isopropanol, ethyl acetate, acetonitrile, and mixtures thereof.

Once a homogenous organic phase is obtained, water is added under conditions permitting formation of a hydrophobic shell about lopinavir-containing oily cores, thus obtaining the nanocapsules. In some embodiments, the volume ratio between the organic phase and the aqueous phase may be between about 2:1 and 4:1.

Such conditions may be, for example, drop-wise or slow addition of the water into the organic phase, mixing at a predetermined speed (for example 500-1500 rpm), etc.

Once a dispersion of nanocapsules is obtained, an aqueous solution comprising the hydrophilic polymer is added under suitable conditions, and the resulting mixed suspension is spray dried thereby obtaining said microparticles.

Spray drying comprises transporting (e.g., delivering, spraying) a colloidal composition (i.e. the mixed suspension) comprising a plurality of the nanocapsules and a microparticle-forming material e.g., the hydrophilic polymeric material, under conditions permitting formation of micronized droplets (i.e. in the sub-micron or micron scale). Said conditions may be, for example, by atomizing, spraying, etc. The size of droplets that are formed by said spray drying determines the (maximal) size (diameter) of the microparticles.

In some embodiments, the method may further comprise drying the microparticles obtained by the spraying method. The drying may be achieved by evaporation of the media solvents by using, for example, lyophillization, thermal drying, reduced pressure, solvent extraction and other techniques.

According to some embodiments, the at least one hydrophilic polymer is an HPMC:Eudragit (poly(methacrylic acid)-ethyl acrylate copolymer) blend. In such embodiments, the aqueous solution may have a pH of about 5.5-6.5 in order to permit satisfactory dissolution of the Eudragit. The pH in the aqueous solution may be, by some embodiments, controlled by the addition of at least one buffer solution.

According to some embodiments, at least one of the following weight ratios is applied in a method of the invention:
(i) the weight ratio of the lopinavir to the oil may be between 1.5:4 and 1.5:6; and/or;
(ii) the organic phase further comprises at least one emulsifier, the weight ratio of the lopinavir to the emulsifier may be between 1:1 and 2:1; and/or
(iii) the organic phase further comprises at least one emulsifier, the weight ratio of the hydrophobic polymer to the emulsifier may be between 2:1 and 4:1; and/or
(iv) the weight ratio of the hydrophobic polymer to the oil may be between at least 3:5; and/or
(v) the weight ratio of the hydrophobic polymer to the solvent may be between 2:1 and 4:1.

According to some embodiments, the organic phase consists of lopinavir, said hydrophobic polymer, said at least one oil, said solvent and optionally at least one surfactant.

According to another aspect, the invention provides a method of preparing the delivery system comprising a protease inhibitor having a log P of between about 2 and 7 as described herein, the method comprising:
mixing (i) at least one oil and the protease inhibitor with (ii) a solution of a hydrophobic polymer in a suitable (e.g. organic) solvent, optionally in the presence at least one surfactant, the weight ratio of the protease inhibitor to the hydrophobic polymer being at least 1:1.5, to thereby form an organic phase;
adding water to said organic phase under conditions permitting the formation a nanocapsules suspension, the nanocapsules comprising a core of said oil formulation and an encapsulation shell comprising said hydrophobic polymer;
mixing said suspension of the nanocapsules with an aqueous solution of at least one hydrophilic polymer to obtained a mixed suspension; and
spray drying the mixed suspension, thereby obtaining microparticles formed of said hydrophilic polymer that embed at least one nanocapsule (the nanocapsule comprising a core comprising the protease inhibitor solubilized in at least one oil, and a shell comprising a hydrophobic polymer, the weight ratio of the protease inhibitor to the hydrophobic polymer being at least 1:1.5).

In another one of its aspects the invention provides a pharmaceutical composition comprising the delivery system of the invention as described herein and at least one pharmaceutically acceptable carrier or excipient.

The “pharmaceutically acceptable carriers” described herein, for example, vehicles, adjuvants, excipients, or diluents, are well known to those who are skilled in the art and are readily available to the public. It is preferred that the pharmaceutically acceptable carrier be one which is chemically inert to the active compound(s) and one which has no detrimental side effects or toxicity under the conditions of use.

The choice of carrier will be determined by a variety of factors. Accordingly, there is a wide variety of suitable formulations of the pharmaceutical composition of the present invention which are suitable for oral delivery. Formulations for oral, parenteral, intravenous, intramuscular, or intraperitoneal administration or formulations for delivery intranasally or by inhalation are merely exemplary and are in no way limiting.

Such pharmaceutical composition is prepared in a manner well known in the pharmaceutical art. In making the pharmaceutical composition of the invention, the aforementioned components are usually mixed with an excipient, diluted by an excipient or enclosed within such a carrier which can be manipulated to the desired form.

In some embodiments, the pharmaceutical composition is in the form suitable for oral administration. In such embodiments, the pharmaceutical composition may be selected from a powder, a tablet, a capsule, a granule, a pill, a lozenge, a troche, a sachet, a chewing gum, and a suspension.

Suspension formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active formulation in an inert base, such as gelatin and glycerin, or sucrose and acacia, emulsions, gels, and the like containing, in addition to the active formulation, such carriers as are known in the art.

The pharmaceutical composition of the invention, independent of the mode of administration, may be engineered or adaptable for immediate release, sustained release, controlled release, slow or fast release, pulsatile release or any other facilitated of a therapeutically effective amount of lopinavor. According to some embodiments, the delivery systems and pharmaceutical compositions comprising then are controlled release compositions, sustained release compositions, or slow release compositions.

Yet a further aspect provides a method of administration of lopinavir to a person in need thereof, comprising orally administering to the subject the delivery system (or a composition comprising the delivery system) of the invention as herein described.

In another aspect, the present disclosure provides a method of treating HIV, comprising administering a therapeutically effective dose of the delivery system (or a composition comprising the delivery system) as described herein to a subject infected by HIV.

As known, the “effective amount” of lopinavir, contained in the delivery system or pharmaceutical composition according to the invention may be determined by such considerations as known in the art. The amount of lopinavir must be effective to achieve the desired therapeutic effect, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, the effective amount depends on a variety of factors including the affinity of the ligand to the receptor, its distribution profile within the body, a variety of pharmacological parameters such as half life in the body, on undesired side effects, if any, on factors such as age and gender, and others.

The delivery system (or pharmaceutical composition comprising it) according to the invention may be used as such to induce at least one effect, e.g., “therapeutic effect”, or may be associated in conjugation with at least one other agent to induce, enhance, arrest or diminish at least one effect or side effect, by way of treatment or prevention of unwanted conditions or diseases in a subject. The at least one other agent (substance, molecule, element, compound, entity, or a combination thereof) may be selected amongst therapeutic agents, i.e., agents capable of inducing or modulating a therapeutic effect when administered in a therapeutically effective amount, and non-therapeutic agents, i.e., which by themselves do not induce or modulate a therapeutic effect but which may endow the nanoparticles with a selected characteristic, as will be further disclosed hereinbelow.

The subject to be treated may be human or non-human.

The delivery system (or a composition comprising microparticles) of the present invention may be selected to treat, prevent or diagnose any pathology or condition, to be treated by administration of lopinavir. The term “treatment” or any lingual variation thereof, as used herein, refers to the administering of a therapeutic amount of the composition or a formulation or a medicament of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

In another aspect, the invention also provides a kit or a commercial package containing the delivery system or pharmaceutical composition of the invention as herein described, and instructions for use. In some embodiments, the composition of the invention or a fraction derived therefrom may be present in the kit in separate compartments or vials.

The kit may further comprise at least one carrier, diluent or solvent useful for the preparation of the composition. The composition may be prepared by the end user (the consumer or the medical practitioner) according to the instructions provided or the experience and/or training of the end-user.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B are SEM micrographs of F[III] formulations of microparticles encapsulating a plurality of NCs containing lopinavir (oleic acid as the oil core) at different magnifications: final microsphere formulations, the scale represents 20 μm (FIG. 1A); following incubation in phosphate buffer 0.2 M at pH 7.4, the scale represents 1 μm (FIG. 1B).

FIG. 2A is a SEM micrograph following 5 min incubation of a MCP of F[II] in PBS (pH 7.4) and (B) Freeze-fractured SEM image a MCP of formulations F[III] after 30 min incubation in PBS buffer (pH 6) at room temperature.

FIG. 2B is a SEM micrograph showing the release or diffusion of PLGA NCs (marked by arrows) from a larger microcapsule, due to the partial dissolution of the external coating polymeric membrane.

FIG. 3 is a SEM micrograph of LPV double-coated NCs F[II] formulation (oleic acid as the oil core) at high magnification, following incubation in HCl/KCl at pH 1.2 over 30 minutes. The microparticles retained their initial structure and the diameter remains in the same range of 1-5 μm, but it can be observed that part of the HPMC polymer dissolved, revealing the presence of Eudragit L-55 fibers and nanocapsules can be detected inside the microparticle.

FIG. 4 is a SEM micrograph of LPV double-coated NCs F[II] formulation (oleic acid as the oil core) at high magnification, following incubation in phosphate buffer 0.2M at pH 7.4 over 30 minutes. No microparticulate structure can be detected owing to complete dissolution of the coating polymer blends. The free spherical nanocapsules can be observed together with polygonal macrocrystals (presumably NaCl salt crystals) precipitated/salt out during the removal of the water from the phosphate buffer.

FIG. 5 shows the releases profile of Lopinavir from microparticulated F[II] nanocapsules at pH=1.2.

FIG. 6 shows the release profile of Lopinavir from microparticulated F[II] nanocapsules at pH=7.4.

FIG. 7 shows the release profile of Lopinavir from microparticulated F[III] nanocapsules at pH=1.2.

FIG. 8 shows the release profile of Lopinavir from microparticulated F[III] nanocapsules at pH=7.4.

FIG. 9 is Lopinavir's release profile from microparticulated F[II] nanocapsules at pH=1.2 (for 2 hours) changed to pH=6.8 (for 6 hours).

FIG. 10 shows average LPV plasma levels in SD male rats following oral administration of 10 mg/kg LPV in different formulations.

### DETAILED DESCRIPTION OF EMBODIMENTS

### 1. Materials and Methods

### 1.1 Materials

Poly(methacrylic acid, Ethyl acrylate) 1:1 (Eudragit® L100-55) was provided by Rohm (Darmstadt, GmbH, Germany). Hydroxypropylmethylcellulose (HPMC) (Methocel E4M Premium) was obtained from Dow Chemical Company (Midland, Mich., USA). Oleic acid (OA) extra pure, DF, NF was purchased from Merck (Darmstadt, Germany). Oleoyl polyoxylglycerides (Labrafil M 1944 CS) was provided by Gattefosse (St. Priest, France). Solutol HS-15 (polyoxyethylene esters of 12-hydroxystearic acid) was provided by BASF (Ludwigshafen Germany). Poly(DL-lactide-co-glycolide) at ratio 50:50, inherent viscosity 0.17 dl/g (PLGA) was purchased from Lactel (Pelham, Ala., USA). Lopinavir (99.1% purity) and Ritonavir (99.8% purity) were purchased from Sequoia Research Products, Pangbourne, United Kingdom.

### 1.2 NC Preparation

The primary NCs were prepared by dissolving 1500 mg OA, 300 mg labrafil M 1944 CS, 300 mg PLGA and 450 mg LPV in 100 ml of acetone. Then, 70 ml of water were slowly added to the oil phase, creating an o/w emulsion, as evidenced by the rapid formation of opalescence in the dispersion medium. The final dried formulation consisted of OA NCs embedded in MCPs and was entitled F[I].

A second formulation was composed of 1500 mg OA, 300 mg labrafil, 450 mg LPV while PLGA was increased to 900 mg. The solvent volumes were tripled to 300 ml acetone to the increase in polymer quantity. Then, 210 ml of water were added slowly to the oil phase, creating an o/w emulsion, as evidenced by the rapid formation of opalescence in the dispersion medium. The final dried formulation consisted of OA NCs embedded in MCPs and was entitled F[II].

A third microparticulate formulation was prepared by dissolving one-third of the F[II] ingredient quantities (i.e. 500 mg OA, 100 mg labrafil, 150 mg LPV and 300 mg PLGA) in 100 ml of acetone. Then, 70 ml of water were added slowly to the oil phase, creating an o/w emulsion. The final dried formulation of OA NCs embedded in MCPs was entitled F[III].

### 1.3 Embedding of NCs in MCPs

The MCPs were formed by microencapsulating the LPV-loaded NCs using the spray-drying technique. 132.26 mg of NaH₂PO₄.H₂O were dissolved in 150 ml of water and pH was adjusted to 6.5 using NaOH 1N. 750 mg of Eudragit L 100-55 were added to this solution and pH was adjusted again in the same manner to 6.5. 100 ml of HPMC solution were prepared by first dispersing 1 g of HPMC in 100 ml of water at about 80° C., and then cooled under stirring to dissolution. The Eudragit L 100-55 at pH of 6.5 was added to the NCs followed by the HPMC solution at room temperature. The acetone was evaporated and the total volume was adjusted to 500 ml with water for obtaining formulations F[I] and FMK while for F[II] the final volume was adjusted to 760 ml. The suspension was spray-dried with a Büchi Mini Spray Dryer B-290 apparatus (Flawil, Switzerland) under the following conditions: Inlet temperature 160° C.; outlet temperature 98° C.; aspiration 100%, pump rate 30% (feeding rate 4 ml/min) and nozzle cleaner 4. The powder was accumulated in the cyclone separator and later collected. The average outlet yield of the process was 42%.

### 1.4 Physicochemical Characterization of Drug-Loaded NCs and Subsequent MCPs

Particle-Size and Zeta Potential Measurements

Nanocapsule size and zeta potential measurements were carried out utilizing a Zetasizer Nano-ZS (Malvern, UK), at 25° C. and using water for HPLC as the solvent.

Drug Content in the Final Dried MCP Formulations

Samples of 10 mg were taken from each formulation. The samples were completely dissolved in 3 ml DMSO, in a volumetric flask under agitation for 1 h and the volume was then adjusted to 10 ml with methanol. 975 μl were withdrawn from each flask and the volume was completed to 1000 μl with internal standard (10 μg) solution of diazepam in methanol. Finally, for all formulations the drug content was determined by injecting 20 μl from each sample into an HPLC device under the following conditions: ACN:H₂O 45:55 mobile phase; 0.8 ml/min flow rate; wavelength 210 nm; XTerra MS C8 5 μm 3.9×150 mm column, purchased from Waters (Milford, Mass., USA). Two calibration curves were constructed from LPV concentrations ranging between 0 and 200 μg/ml and internal standard diazepam at a concentration of 10 μg/ml. The samples of the first calibration curve were dissolved in methanol and the samples of the second curve were dissolved in DMSO:MeOH 3:7, as were the formulation samples. The calculated recovery percentage was 100%.

### 1.5 Morphological Evaluation

Optical and Scanning Electronic Microscopy (SEM) Studies of MCPs

Morphological evaluation of spray-dried NC-loaded microspheres was carried out using High-Resolution Scanning Electron Microscope (Sirion, HR-SEM; FEI Company, The Netherlands). The specimens were fixed on an SEM-stub using double-sided adhesive carbon tape or alternatively, suspensions were poured into a cover glass to evaporate the water medium. After evaporation, standard coating by Au—Pd sputtering (Pilaron E5100) under vacuum made the specimen electrically conductive. To evaluate the effect of incubation pH buffers, the following procedure was carried out: 10 mg of formulation were soaked in 2 ml of PBS pH 7.4; 200 μl of the suspensions were poured into a glass clock plate to evaporate the water medium; following evaporation the dry formulations were collected for SEM evaluation.

### 1.6 In-Vitro Studies

LPV In-Vitro Release Profile Experiment

A solubility test was performed prior to the in-vitro release profile experiment to ensure that sink conditions would prevail by adding 10 mg LPV to 10 ml of 0.1% Tween 80 pre-warmed to 37° C. in a beaker [17]. The pH was adjusted to 7.4 and the Tween solution was kept at 37° C. and rotated at 150 rpm for 3 h. Then, 5 ml aliquots were taken and filtered using a 0.45 μm PVDF filter and diluted with methanol to a final dilution factor of 10. LPV solubility following 3 h incubation in 0.1% Tween was 72.7 μg/ml as determined by the above-described HPLC technique.

LPV in-vitro release kinetic experiments were carried out using the apparatus (VanKel VK7000 with a VK 750D pump) comprised of six vessels containing 300 ml of 0.1% Tween 80 at 37° C. with USP paddles rotating at a speed of 50 rpm. The three formulations (F[I]-F[III]) were added to the different vessels at a LPV quantity equivalent to maintain sink conditions (10% of the pre-determined solubility). The pH was adjusted to 7.4 and 5 ml aliquots were taken following 0, 15, 30, 45, 60, 90, 120, 180 min incubation. The samples were filtered using 0.45 μm PVDF filter and 500 μl from the filtrate was diluted in 500 μl methanol. These samples were comprised of free and encapsulated LPV, since LPV NCs diameter size is about 200 nm and the NCs can diffuse via 0.45 μm membrane pores. The remaining filtrate was then transferred to diafiltration membrane vivaspin 6 (MWCO 300,000 purchased from Sartorius Stedim Biotech) and centrifuged for 15 min at 4000 rpm. 500 μl from the vivaspin filtrate were diluted with methanol in the same manner. These samples were comprised solely of free LPV. All samples were analyzed using HPLC. The experiment was not performed at pH 1.2 due to precipitation of LPV and low solubility below HPLC detection limit.

### 1.7 In-Vivo Studies

Pharmacokinetic Studies in Rats

All the animal studies were carried out in accordance with the rules and guidelines concerning the care and use of laboratory animals and were approved by the local Ethical Committee of Laboratory Animal Care at The Hebrew University of Jerusalem (Approval No: MD-09-12092-3).

Sprague-Dawley male rats weighing 270-300 g were used and were separated randomly into seven groups with 4-8 animals per group to evaluate the biofate of LPV in different formulations. The animals were housed in SPF conditions, fasted overnight (12-14 h prior to the experiment) with free access to water. The animals were given a dose of 10 mg/kg LPV with or without RTV, always at the same ratio as the Kaletra® administered orally, using gavage. The groups were orally administered according to the following experimental conditions: 1 ml of Kaletra® oral solution (LPV 80 mg/RTV 20 mg/ml) prepared by diluting the purchased commercial solution with 1:1:1 ethanol:propylene glycol:DDW to a concentration of LPV 3 mg/RTV 0.75 mg/ml; and microparticulate formulations, F[I]-F[III] prepared by dispersing the NCs embedded in MCPs in DDW to the final concentration of 40 mg/ml. Blood samples (400-500 μl) were withdrawn from the tail vein at 0, 0.5, 1, 2, 4, 8, 12 and 24 h. Saline solution was given to the animals after 30 min and then again after 3 h. The blood samples were collected in heparin containing tubes. The samples were immediately centrifuged at 10,000 rpm for 5 min, after which 200-300 μl of plasma samples were transferred to new tubes and stored at −80° C. until further analysis by LC-MS/MS as described below.

Plasma Level Determination

The blood samples were treated by protein precipitation in methanol, following 15 min centrifuge at 10,000 rpm. 25 ng of quinoxaline (QX) dissolved in methanol were added to each sample, as an internal standard. The supernatant layer was collected and the samples were injected into an LC-MS/MS device under the following conditions: using a Phenomenex Kenetex column (RP-C18, 50×2.1 mm, 2.6 μm, 100 A) in gradient mode; the mobile phase consisted of A=methanol/formic acid 99.9/0.1 and B=water/formic acid 99.9/0.1; the A:B ratio was 58:42 at t=0 min, during the first 1.5 min the ratio changed gradually to A:B 80:20 and remained steady until 2.1 min. The ratio returned to A:B 58:42 rapidly from 2.1-2.15 min and remained constant until the end of the run at 5 min for the purpose of system cleaning and stabilization. The flow rate was maintained at 0.35 ml/min, and the column temperature was maintained at 35° C. LC-MS/MS analysis was performed with a thermo scientific Accela HPLC system coupled with a TSQ Quantum Access MAX detector in positive ionization mode. Detection and quantification were carried out by multiple-reaction monitoring with transitions from m/z 629.3 to 156 for LPV and from m/z 313.1 to 246 for QX. The tested samples were quantified against a calibration curve in the range of 0-100 ng/ml. The correlation coefficient values were higher than 0.99 indicating that good linearity, accuracy and specificity were achieved.

### 1.8 Ex-Vivo Antiviral Activity of Rat Serum Samples Following Oral Administration of Various Lopinavir Formulations

Sprague Dawley male rats weighing 300-350 g were used in this specific study and separated randomly into three groups (three animals per group) to evaluate the biofate of LPV in different formulations. The animals were housed in SPF conditions, fasted overnight (12-14 h prior to the experiment) with free access to water. The animals were given orally a dose of 10 mg/kg LPV in different nano/microparticulate formulations (F[II]; F[III]) and Kaletra® using gavage. Blood samples (400-500 μl) were withdrawn from the tail vein at 0, 1, 2, 4, and 8 h. Saline solution was given to the animals after 30 min and then again after 3 h. The blood samples were collected in tubes and stand for 15 minutes and then immediately centrifuged at 5,000 rpm for 10 min, after which 200-300 μl of serum samples were transferred to new tubes and stored at −80° C. until further analysis.

### 2. Results

### 2.1 Morphological Evaluation

The average diameter measurements of the NCs formed for F[I] were 236 nm. The NCs exhibited a narrow size-distribution range as reflected by the low value of the polydispersity index (PDI) in all formulations. The average zeta potential was −46 mV for all formulations. The drug content in the final dried OA NC-based MCP formulations was between 95 and 105% from the initial theoretical content of the formulations. More specifically, the content (w/w) of F[I] was 9.9%, F[II] 9.6% (Table 1) and F[III] 5.5% (Table 2) w/w, respectively.

Six different batches of F[II] were prepared and the mean diameter size of the NCs prior to embedding in the MCPs ranged from 200 to 280 nm and the Zeta Potential value ranged from −45.8 to −51.5 mV (Table 1). Eight batches were prepared from F[III]; as noted from the data presented in Table 2, the mean diameter of the NCs prior to embedding in the MCPs ranged from 290.1 to 537.6 nm and the Zeta Potential value ranged from −36.3 to −40.4 mV. As can be seen, reproducible results were obtained. It is noted that the batches from each formulation were pooled together for the following evaluations.

TABLE 1
Summary of the physicochemical parameters of six batches of
formulation F[II]

| | | | Zeta | Batch | Yield | LPV |
|---|---|---|---|---|---|---|
| | Size | | potential | weight | of batch | content |
| Batch # | (nm) | PDI | (−mV) | (mg) | (%) | (μg/mg) |
| FII-1 | 200 ± 6.8 | 0.39 | 45.8 ± 0.59 | | | |
| FII-2 | 280.6 ± 1.9 | 0.42 | 47.7 ± 0.6 | 2459.3 | 48.7 | 95.47 |
| FII-3 | 251.8 ± 3.6 | 0.412 | 50.8 ± 0.86 | 2384 | 47 | 97.93 |
| FII-4 | 269.8 ± 2.7 | 0.39 | 50.3 ± 0.38 | 2605.6 | 51.6 | 93.49 |
| FII-5 | 257.3 ± 3.6 | 0.41 | 47.4 ± 1.21 | 2776 | 55 | 94.44 |
| FII-6 | 201.3 ± 3.6 | 0.39 | 51.5 ± 0.40 | 2536 | 50.35 | 102.23 |

TABLE 2
Summary of the physicochemical parameters of eight batches of
formulation F[III]

| | | | Zeta | Batch | Yield | LPV |
|---|---|---|---|---|---|---|
| | Size | | potential | weight | of batch | content |
| Batch # | (nm) | PDI | (−mV) | (mg) | (%) | (μg/mg) |
| FIII-1 | 391 ± 1.1 | 0.2 | 38.9 ± 1.0 | 1718.2 | 59 | 51.61 |
| FIII-2 | 537.6 ± 29.6 | 0.32 | 38.6 ± 0.3 | 1792 | 59 | 53.66 |
| FIII-3 | 353.4 ± 23.2 | 0.28 | 36.3 ± 0.2 | 1674 | 57 | 48.03 |
| FIII-4 | 400.2 ± 13 | 0.331 | 36.9 ± 0.4 | 1734 | 59 | 52.12 |
| FIII-5 | 336.3 ± 17.1 | 0.29 | 37.8 ± 0.5 | 1727 | 59 | 55.19 |
| FIII-6 | 315.2 ± 15.6 | 0.328 | 38.1 ± 0.1 | 1750 | 60 | 48.55 |
| FIII-7 | 290.1 ± 13.1 | 0.385 | 40.3 ± 3.2 | 1736 | 59 | 56.21 |
| FIII-8 | 294.7 ± 14.6 | 0.335 | 40.4 ± 0.4 | 1658 | 56 | 50.08 |

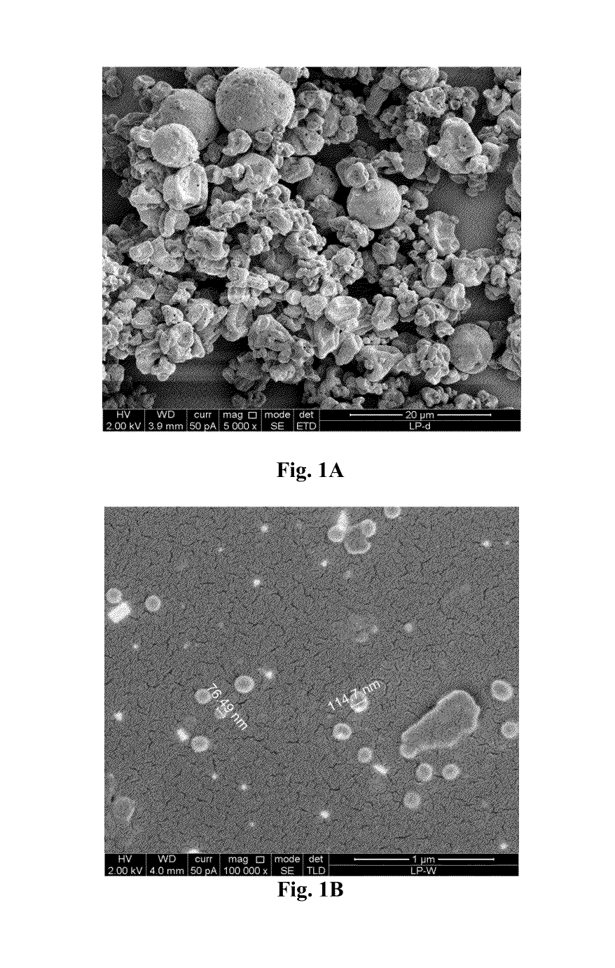

SEM micrographs of the final formulations, LPV-NC-loaded MCPs at different magnifications are depicted in FIGS. 1-3. It can be seen from FIG. 1A-B that formulations from F[III] comprised of spherical MCPs ranging qualitatively in size from 1-10 μm as estimated from the SEM observations. From FIG. 1A it can be observed that some of the MCPs of the different formulations lost their shape, and collapsed areas, due to MCP internal void volumes attributed to the vacuum needed to operate the SEM apparatus. The MCP matrices are composed mostly of HPMC and Eudragit L100-55, which is readily soluble only above pH 5.5. In FIG. 1B, it was not possible to distinguish any regular morphological structures in the same formulations following incubation of the spray-dried MCPs prepared with Eudragit L and HPMC coating polymers in the release medium pH 7.4 for 1 h. Both polymers dissolved and no defined structure could be identified. This suggests that the NCs are expected to easily be released by such a delivery system since individual homogeneous NCs can be identified at the size of about 100 nm (FIG. 1B).

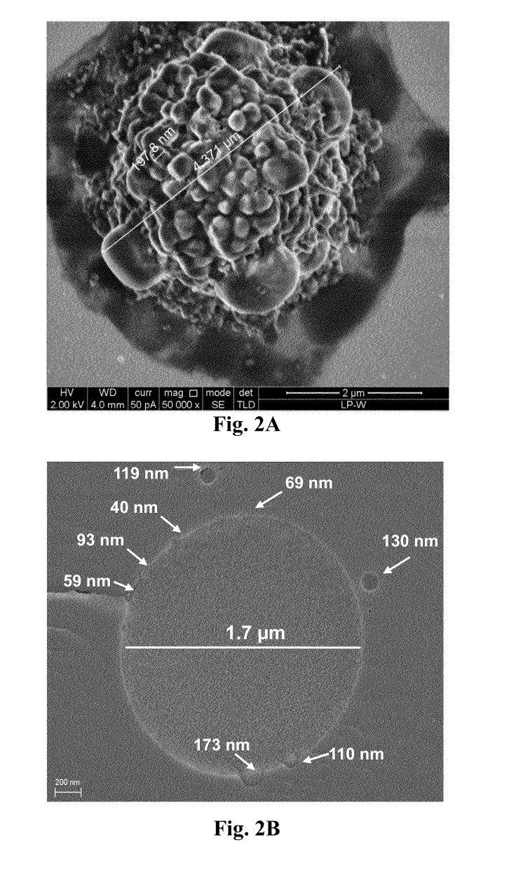

Furthermore, for the purpose of confirmation that NCs are released from the MCPs following incubation in PBS over short periods of time, two additional independent experiments were carried out and the morphological results are depicted in FIGS. 2A-2B; SEM images following 5 min incubation of a F[II] MCP in PBS (pH 7.4) (FIG. 2A) and a freeze-fractured SEM image of a F[III] MCP after 30 min incubation in PBS buffer (pH 6) at room temperature (FIG. 2B). As can clearly be seen in FIG. 2A, the external coat dissolves to reveal the presence of the internal NCs prior to their separation with mean diameter size ranging from 200 to 400 nm. FIG. 2B captures the release or diffusion of PLGA NCs (arrows) from a larger microcapsule due to the partial dissolution of the external coating polymeric membrane.

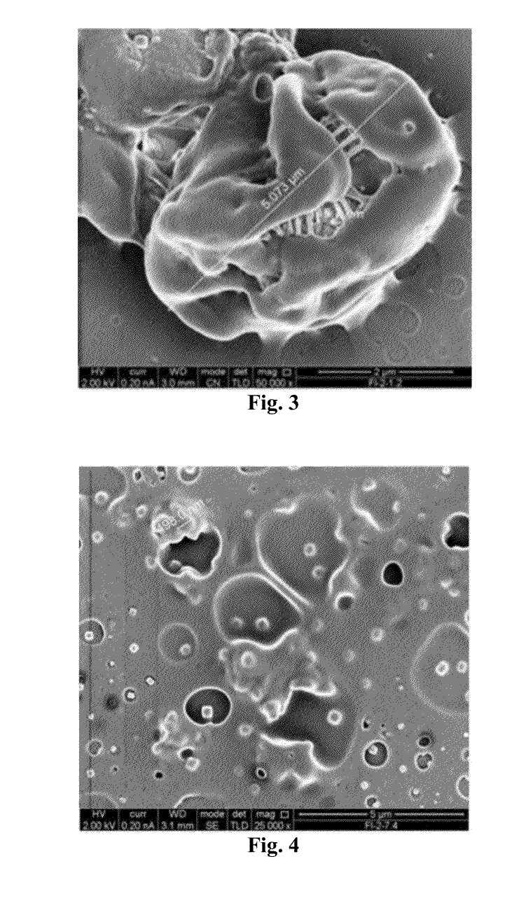

FIG. 3 shows SEM micrographs of LPV double-coated NC formulations, F[II] (OA as the oil core) at high magnifications, following incubation in HCl/KCl at pH 1.2 for 30 min. The MCPs retained their initial structure and the diameter remained in the same range of 1-5 μm but it can be observed that part of the HPMC polymer dissolved revealing the presence of Eudragit L-55 fibers and NCs inside the MCP matrix. Contrastingly, FIG. 4 shows SEM micrographs of LPV double-coated NCs formulations, F[II], (OA as the oil core) following incubation in phosphate buffer 0.2M at pH 7.4 for 30 min at high magnification and no MCP structure can be detected owing to complete dissolution of the coating polymer blends. However, the free NCs can be observed together with macro crystals which originated from the removal of the water from the phosphate buffer, confirming the results of F[III] in FIG. 1B.

### 2.2 In-Vitro Release Profile Experiments

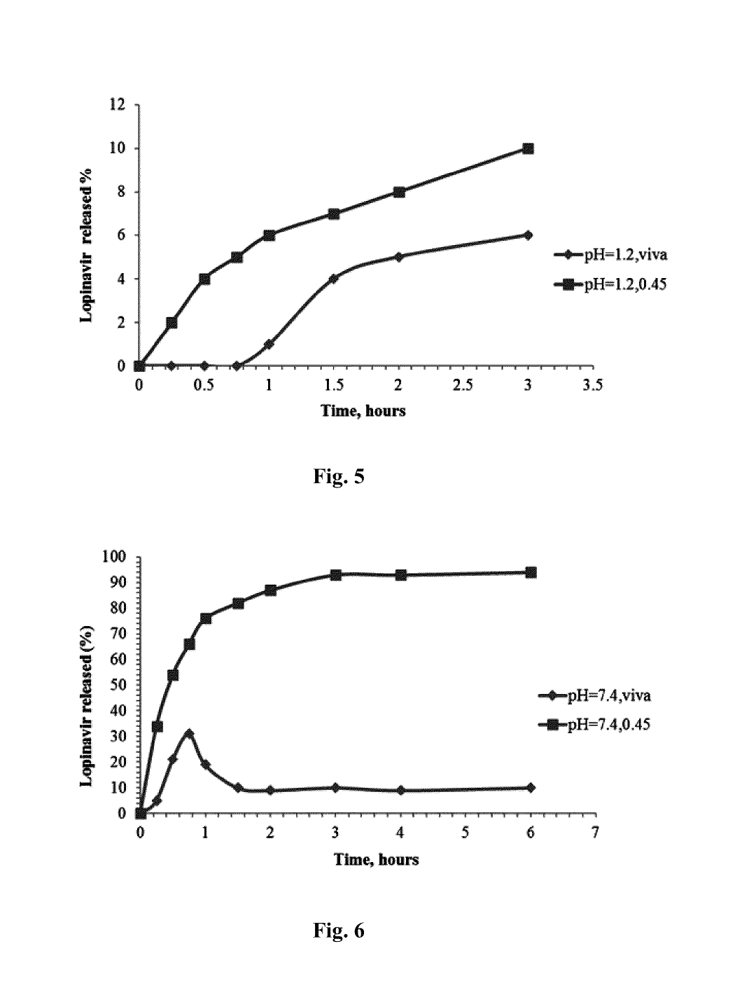

The LPV in-vitro release profile for the different formulations is provided in FIGS. 5-9. The graphs display both the total amount of LPV released from the MCPs, as free dissolved molecules and the LPV molecules still entrapped in the NCs, as detected following filtration of the dissolution samples through 0.45 μm PVDF filters (dimond-shaped points), as well as the free dissolved LPV molecules fraction only, as detected following a second filtration using 300,000 MW cut off vivaspin membranes which retained the NCs (square points). Following immersion in infinite volume, the MCPs dissolve and release both free LPV molecules and LPV entrapped in NCs. In fact, for F[I] after 45 min almost 100% of the LPV was released from the MCPs and NCs at pH 7.4. It was therefore suggested that the NC coating was too thin and could not retain the entrapped LPV once sink conditions prevail.

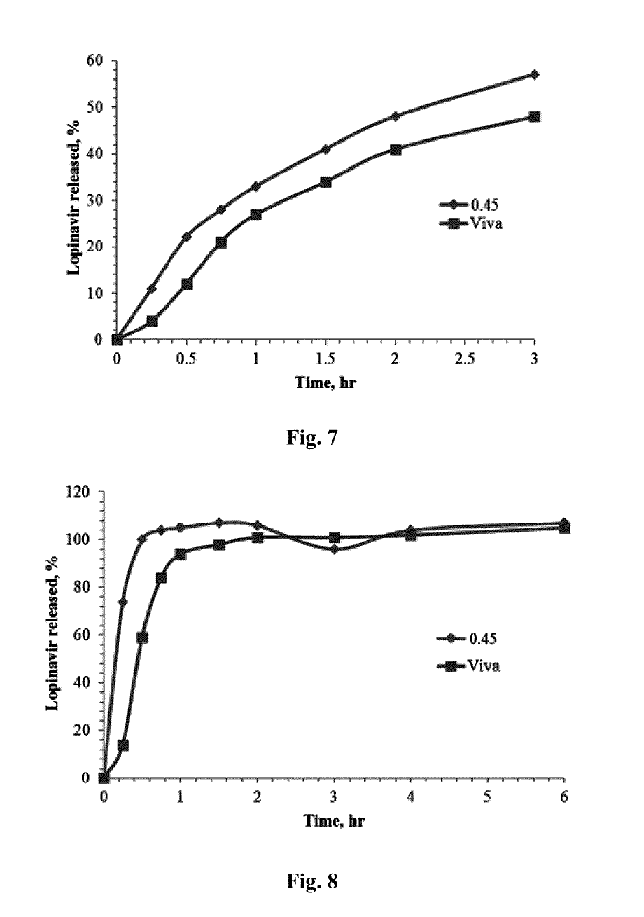

Indeed, when observing the results for F[II] and F[III], formulations designed with thick NC coating and prepared without solvent excess, it can be seen that the release of LPV from the NCs decreased at pH 1.2 (FIG. 5 and FIG. 7 for F[II] and F[III], respectively). Almost no free LPV was released from F[II] and less than 10% of the LPV-entrapped NCs were released within 3 h. For F[III], about 30% of free LPV and more of 40% of free LPV and LPV-entrapped NCs were released within 2 h (FIG. 7).

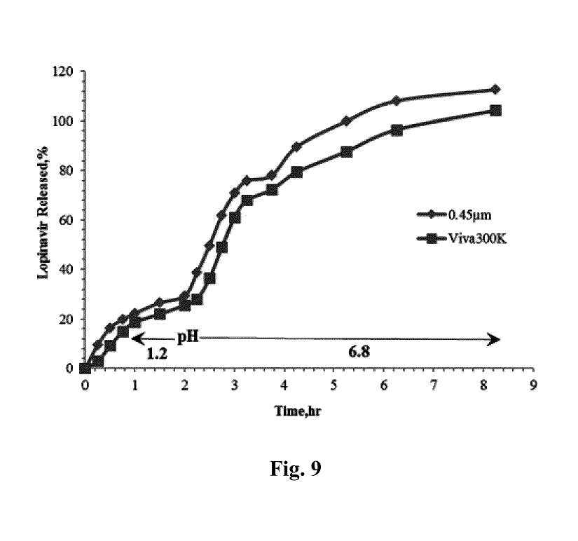

Furthermore, the in-vitro release kinetic behavior at pH 7.4 was different. For F[II] it is noted that most of the LPV-entrapped NCs were released within 2 h but almost no free LPV was released from such NCs (FIG. 6). For F[III], as shown in FIG. 8, both the curves are similar, indicating that the NCs could not retain the LPV within their oil core since most of the LPV was released in less than 2 h. When the pH was increased from 1.2 to 6.8 for F[II] in the same flasks (FIG. 9), the LPV release augmented within 1 h and reached 60% due to the dissolution of the Eudragit L-55 MCP coating and exposure to the release medium of the NCs, which were then unable to retain the LPV within their oil cores. Nevertheless, it is expected that LPV encapsulated in F[II] should be better protected in the lumen gut than by F[III].

### 2.3 Rat Pharmacokinetic Studies Analysis

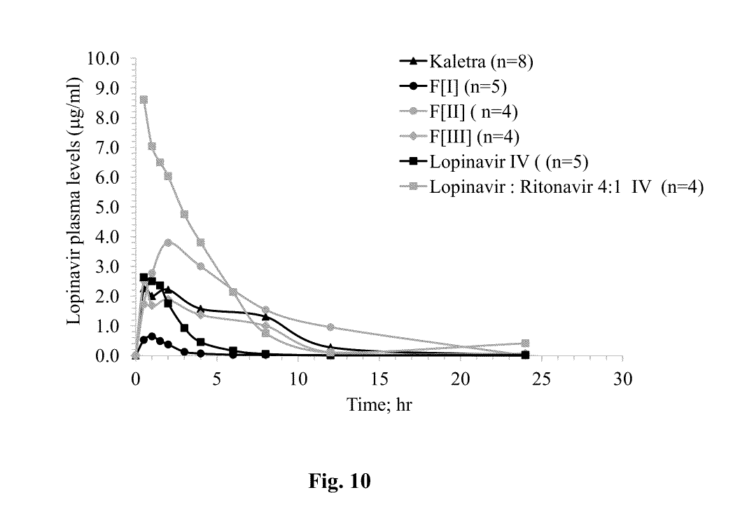

The change in LPV plasma concentrations following intravenous (i.v.) administration of LPV alone and combined with RTV in an aqueous solution and the pharmacokinetic (PK) profile of LPV following oral administration of various formulations is depicted in FIG. 10. The calculated PK parameters for all the formulations are displayed in Table 3. The absolute bioavailability (F) of LPV at all dosage forms was calculated relative to the AUC (area under the curve) yielded by i.v. administration of LPV and RTV at the ratio of 4:1 (F=1.0) since the same combination of LPV and RTV, when administered orally, is considered the standard of PI care for HIV-linfected patients. It can be seen from FIG. 10 and Table 3 that LPV bioavailability increased almost 5-folds following i.v. administration of the combination LPV:RTV as compared to LPV alone, as evidenced by the AUC values (38562±7923 versus 8004±1215 h·ng/ml, p<0.001). Co-administration of RTV i.v. also increased extrapolated plasma concentration at T₀ from 2777±203 to 10591±2908 ng/ml respectively, decreased CL from 1269±210 to 260±51 ml/h/kg respectively and extended the half-life from 1.2±0.2 to 2.2±0.8 h respectively (Table 3).

Furthermore, LPV oral administration elicited a low absolute bioavailability compared to i.v. LPV solution (24%) and much less compared to LVR:RTV i.v. (5%). The oral administration results also confirm the significant effect of RTV, since Kaletra® oral solution increased LPV AUC values almost 9-folds compared to oral LPV. However, when comparing the PK parameters: AUC, Cₘₐₓ,T_{1/2} and CL of the formulation F[I] and to LPV oral solution, clearly there is no advantage to this formulation, suggesting that the nanoencapsulation was unable to retain LPV in the formulation under the described experimental conditions.

The PLGA coating concentration was increased in F[II] and the PK profile (FIG. 10) and AUC (Table 3) were markedly enhanced. The oral administration of formulation F[II], which contains the same ratio of LPV:PLGA but prepared with three times more acetone and only 50% more water as compared to F[III], not only resulted in a 2-folds AUC value increase compared to Kaletra®, but also achieved almost similar AUC values as i.v. administration of LVR:RTV 4:1 (31477±4871 versus 38562±7923 h×ng/ml).

TABLE 3
Average PK parameter values (mean ± SD)
following oral/IV administration of 10 mg/kg
LPV in different formulations; N = 4-8.

| | Cₘₐₓ | T_{1/2} | AUC | |
|---|---|---|---|---|
| Formulation | (ng/ml) | (h) | (h × ng/ml) | F |
| LPV:RTV 4:1 i.v. | 10591 ± 2908 | 2.2 ± 0.8 | 38562 ± 7923 | 1 |
| (n = 4) | | | | |
| LPV i.v. (n = 5) | 2777 ± 203 | 1.2 ± 0.2 | 8004 ± 1215 | 0.21 |
| LPV oral so. | 780 ± 246 | 4.8 ± 0.9 | 1908 ± 324 | 0.05 |
| (n = 4) | | | | |
| Kaletra ® oral | 2593 ± 408 | 2.3 ± 0.95 | 18040 ± 2942 | 0.47 |
| sol. (n = 8) | | | | |
| F[I] oral | 694 ± 422 | 4.8 ± 1.9 | 1529 ± 972 | 0.04 |
| formulation | | | | |
| (n = 5) | | | | |
| F[II] oral | 3793 ± 1503 | 2.15 ± 0.3 | 31477 ± 4871 | 0.82 |
| formulation | | | | |
| (n = 4) | | | | |
| F[III] oral | 2655 ± 1033 | 4.2 ± 0.9 | 15173 ± 1072 | 0.39 |
| formulation | | | | |
| (n = 4) | | | | |

### 3. Discussion

It is well established that LPV exhibits low oral and variable bioavailability in rats and humans when given alone owing to extensive gut and systemic CYP3A4 metabolism as well as LPV efflux by transporters such as P-gp and Multidrug Resistance Protein. Co-administration of low-dose RTV particularly inhibits LPV metabolism by CYP3A4 as already described [18], in which it was reported on a marked increase in AUC of LPV in the presence of RTV by a factor of 4 in rats, and 20 in humans upon i.v. and oral administration respectively [19, 20]. The comparative findings herein show similar results, as i.v. bolus of LPV and RTV at a ratio of 4:1 resulted in an increase of 4.8-fold in AUC compared to LPV IV in rats (Table 3); as well as by the oral administration results since an oral dosage of LPV compared to Kaletra® oral solution which resulted in an AUC decrease from 18040±2942 to 1908±324 h×ng/ml (p<0.01) and a 3-fold decrease in Cₘₐₓ (Table 3).

Although the Kaletra® therapeutic efficacy is well established, the concerns raised by the side effects attributed to the presence of RTV required a different approach to improve LPV bioavailability. According to the present invention, a drug-delivery system based on double-coated NCs entrapping LPV and embedded in MCPs was developed in order to bypass the P-gp efflux and protect the drug from CYP3A pre-systemic metabolism, without co-administration of RTV. Three formulations according to the present invention were designed and demonstrated to have good chemical stability, similar physicochemical properties with negative zeta potential (−36 mV to −46 mV), and NCs diameter size of 170-236 nm. The NCs were shown to be released from the MCPs following 1 h incubation at pH 7.4 and were easily detected by SEM imaging owing to the complete and partial dissolution of HPMC and Eudragit L polymers forming the MCP matrices at such pHs and incubation times. The NCs qualitative average diameters ranged from 40-350 nm depending on the experimental conditions. These findings were further confirmed by the in-vitro release kinetic results.

As can be seen in FIGS. 8-9, depicting the LPV release profile from RIM and F[II], respectively, the total LPV release profile is similar to the dissolution profile of free LPV molecules. Following dissolution in an infinite volume at pH 7.4, the MCPs dissolved and released both free LPV molecules and LPV entrapped in NCs relatively rapidly since within 2 h almost 100% and 70% of the LPV was released from the MCPs and NCs from RIM and F[II]. These findings suggested that a thin NC coating is not able to retain an encapsulated drug under sink conditions. However, formulation F[II] released LPV relatively more slowly than the other formulations especially RIM seemingly due to a thicker NC coating, and, more importantly, despite a higher NC-load capacity in the MCPs resulting in a final drug content of 9.66% compared to 5.52% for the F[III] formulation. Despite the higher load of NCs in the MCPs no rapid LPV release from the NCs in the gut lumen occurred even if sink conditions prevail.

Regardless of sink conditions, the MCPs prevented rapid drug release and allowed NCs to adhere to the intestinal mucosa (as observed in a previous study where Nile red NCs were shown to adhere to the enterocyte membrane in the jejunum and enter the cytoplasm of the enterocytes 30 min following oral administration of the MCPs [12]). Therefore, in systems of the present invention, it can be envisioned that when LPV is entrapped in NCs that penetrate the enterocytes, it is protected from efflux by P-gp and extensive CYP metabolism and remains available for absorption into the circulation.

When the formulations were administered orally to Sprague-Dawley male rats, the significant differences in the PK profiles were remarkable and it was suggested that the preparation method of the formulation determines its PK properties and in-vivo behavior.

These differences were not entirely revealed by the in-vitro release kinetic experiments. Kaletra® oral solution bioavailability improved almost 9-fold compared to the LPV oral solution whereas F[I] elicited a poor LPV absorption and a low bioavailability similar to that of the LPV solution. It was suggested that this was not due to the high drug content (10%, w/w) but rather due to the decreased thickness of the NC coating which contributed to a more rapid release of LPV from the NCs in the intestine despite the identical coating polymer composition of the MCPs, behaving similarly to the LPV solution. Thus, free LPV and LPV released from the NCs under the physiological sink conditions were either effluxed by the P-gp or metabolized by CYP3A4, resulting in poor oral bioavailability

A significant improvement in LPV absorption profile was achieved with F[II] and F[III], as compared to F[I] since the AUC average value of F[III] and F[II] increased by 8 and 16-fold compared to F[I] respectively, although such an improvement could not have been expected based on the in-vitro release kinetic experimental results (FIGS. 8 and 9). Although correlation between in-vitro release model and in-vivo behavior may be difficult to achieve with Pg-p substrate drugs, in-vitro release profile results can be used as an in-process-control parameter for such formulations. The improvement in the oral bioavailability noted with F[II] was attributed to a better protection of LPV from the biochemical barriers resulting in an increase in oral absorption. Thus, the manufacturing process of the NCs and the loading extent of NCs in MCPs, which is reflected by the drug content values (9.66 and 5.52% for F[II] and F[III] respectively), can markedly affect the biological performance of such oral formulations.

The preparation method for both formulations was similar except for the quantity of NC excipients, LPV, where acetone was increased 3-fold for F[II] compared to F[III] while the water was increased only by 50% in order to improve the drug entrapment in the NCs and the NC-loading in the final MCP-formulation. As can be seen, F[II] elicited the highest absorption profile, and the AUC value increased 2-fold compared to the commercial product Kaletra®. Moreover, the oral bioavailability of F[II] was close (82%) to the value yielded by the i.v. administration of LPV:RTV 4:1 showing that LPV in rats can be effectively protected from the CYP degradation when encapsulated not only in the gut and enterocytes but also in the systemic circulation, suggesting that LPV-loaded modified nanoparticulate structures reached the circulation.

These findings suggest that initially the LPV-loaded NCs, while transiting via the intestinal mucosa, not only did not release the active ingredient within the enterocytes but were subjected to physiological modifications that allowed these nanostructures to reach the systemic circulation while continuing to protect LPV from the detrimental CYP effect in the blood. In view of these findings, it is expected that LPV in plasma will be released progressively from the NCs and will exert on it biological activity.

Finally, the marked LPV enhancement exposure following oral administration of the formulation F[II] suggests that NCs both circumvent P-gp and protect the drug from CYP3A intestinal and systemic metabolism and it is likely that the lymphatic system is involved. If indeed the lymphatic system is involved in the uptake of nanoencapsulated LPV to the circulation, this finding is envisaged to have clinical significance since the HIV retrovirus is known to accumulate and even reside in the lymphatic-system.

1. A controlled-release delivery system for oral delivery of lopinavir, the delivery system comprising at least microparticle formed of a hydrophilic polymeric matrix, and embedding at least one nanocapsule, the at least one nanocapsule comprises a core comprising lopinavir solubilized in at least one oil, and a shell comprising a hydrophobic polymer, the weight ratio of the lopinavir to the hydrophobic polymer being at least 1:1.5.

2. The delivery system of claim 1, comprising a plurality of said nanocapsules.

3. The delivery system of claim 1, wherein said core consists of lopinavir and said at least one oil, optionally further consisting at least one surfactant.

4. The delivery system of claim 1, wherein the hydrophilic polymer is selected from the group consisting of poly(methacrylic acid), ethyl acrylate, polyols, polycarbohydrates, hydroxypropylmethyl cellulose (HPMC), hydroxymethyl cellulose, hydroxypropylmethylcellulose phthalate (HP55), cellulose acetate phthalate, carboxy-methylcellulose phthalate, shellac, zien, and copolymers and mixtures thereof.

5. The delivery system of claim 1, wherein said hydrophilic polymeric matrix comprises an HPMC:Eudragit (poly(methacrylic acid)-ethyl acrylate copolymer) blend.

6. The delivery system of claim 1, wherein said microparticle having an average diameter of between about 0.5 and 20 μm.

7. The delivery system of claim 1, wherein said oil is selected from the group consisting of liquid fatty acids, esters thereof and any mixture thereof.

8. The delivery system of claim 1, wherein said oil is selected from the group consisting of oleic acid, octanoic acid and mixtures thereof.

9. The delivery system of claim 1, wherein said hydrophobic polymer is selected from the group consisting of lactic acid, poly(D,L-lactic-co-glycolic acid) (PLGA), poly(D,L-lactic acid) (PLA), poly(ε-caprolactone), poly(2-dimethylamino-ethylmethacrylate) homopolymer, poly(2-dimethylamino-ethylmethacrylate)-b-poly(ethyleneglycol)-α-methoxy-ω-methacrylate copolymers, polycyanoacrylates and combinations thereof and their PEGylated derivatives.

10. The delivery system of claim 1, wherein the nanocapsules have an average diameter of between about 40 and 500 nm.

11. The delivery system of claim 1, wherein the microparticle comprises between about 1 and 10 wt % of lopinavir.

12. The delivery system of claim 1, wherein in the nanocapsule
(i) the weight ratio of the lopinavir to the oil being between 1.5:4 and 1.5:6; and/or;
(ii) the core further comprises at least one emulsifier, the weight ratio of the lopinavir to the emulsifier being between 1:1 and 2:1; and/or
(iii) the core further comprises at least one emulsifier, the weight ratio of the hydrophobic polymer to the emulsifier being between 2:1 and 4:1; and/or
(iv) the weight ratio of the hydrophobic polymer to the oil being at least 3:5.

13. The delivery system of claim 12, wherein in the nanocapsule the weight ratio of lopinavir to hydrophobic polymer to oil is 1.5:3:5.

14. A method of preparing the delivery system of claim 1, comprising:
mixing (i) at least one oil and lopinavir with (ii) a solution of a hydrophobic polymer in an organic solvent, optionally in the presence at least one surfactant, the weight ratio of the lopinavir to the hydrophobic polymer being at least 1:1.5, to thereby form an organic phase;
adding water to said organic phase under conditions permitting the formation of a suspension of nanocapsules;
mixing said suspension of nanocapsules with an aqueous solution of at least one hydrophilic polymer to obtained a mixed suspension; and
spray drying the mixed suspension, thereby obtaining said microparticles.

15. The method of claim 14, wherein said organic solvent is selected from the group consisting of acetone, methanol, ethanol, isopropanol, ethyl acetate, acetonitrile, and mixtures thereof.

16. The method of claim 14, wherein
(i) the weight ratio of the lopinavir to the oil being between 1.5:4 and 1.5:6; and/or;
(ii) the organic phase further comprises at least one emulsifier, the weight ratio of the lopinavir to the emulsifier being between 1:1 and 2:1; and/or
(iii) the organic phase further comprises at least one emulsifier, the weight ratio of the hydrophobic polymer to the emulsifier being between 2:1 and 4:1; and/or
(iv) the weight ratio of the hydrophobic polymer to the oil being between at least 3:5; and/or
(v) the weight ratio of the hydrophobic polymer to the solvent is between 2:1 and 4:1.

17. The method of claim 16, wherein in the weight ratio of lopinavir to hydrophobic polymer to oil to acetone in the organic phase is 1.5:3:5.

18. The method of claim 14, wherein the organic phase consists of lopinavir, said hydrophobic polymer, said at least one oil, said solvent and optionally at least one surfactant.

19. A method of treating HIV, comprising administering a therapeutically effective dose of the delivery system of claim 1 to a subject infected by HIV.

20. The method of claim 19, wherein the core of the nanoparticle consists of lopinavir and said at least one oil, optionally consisting at least one surfactant.
